(19) United States
(12) Patent Application Publication
(10) Pub. No.: US 2012/0328689 A1
(43) Pub. Date: Dec. 27, 2012

(54) DEODORIZING PAPER AND METHOD

(76) Inventors: Nigel Flynn, Flowery Branch, GA (US); Mark Caligaris, Greenville, SC (US); Taylor Renee Kopacka, Alpharetta, GA (US)

(21) Appl. No.: 13/583,120

(22) PCT Filed: Mar. 8, 2011
(86) PCT No.: PCT/US11/27583

§ 371 Date: Sep. 6, 2012

Related U.S. Application Data
(60) Provisional application No. 61/311,664, filed on Mar. 8, 2010.

(51) Int. Cl. A61L 9/012; B05D 7/00

(52) U.S. Cl. 424/443; 427/372.2

(57) ABSTRACT

A deodorizing paper substrate is provided that has a base sheet that incorporates between 3%-15% of sodium bicarbonate. The deodorizing particles remove malodors from any environment. An associated method of manufacture is also provided.

### CROSS REFERENCE TO RELATED APPLICATIONS

This application claims the benefit of U.S. Application Ser. No. 61/311,664 filed on Mar. 8, 2010 and entitled, “Lint and Clothes Storage Apparatus and Process”. U.S. Application Ser. No. 61/311,664 is incorporated by reference herein in its entirety for all purposes.

### FIELD OF THE INVENTION

The present invention relates generally to a deodorizing substrate. More particularly, the present application involves a deodorizing substrate that is capable of removing undesired odors from an article. An associated method of manufacture is also provided.

### BACKGROUND

The use of baking soda (sodium bicarbonate) as an odor absorbing agent is well known. Open boxes of baking soda have been used to control odor in refrigerators, cabinets and other enclosed spaces.

U.S. Pat. No. 5,046,604 to Forhetz et al. teaches an odor absorbing liner that uses pouches of odor absorbing particulates which are stitched between sheets in a quilt-type fashion. The odor absorbing particles are loose in the pouches and if substrate becomes torn, the particles can be released.

It is also know in the art to incorporate into a cellulose paper product sodium bicarbonate by adding the sodium bicarbonate into an aqueous suspension of paper making fibers as set forth in U.S. Pat. No. 7,462,260 to Spence et al. While the Spence et al. patent is directed to absorbing unpleasant odors inherent in the paper product when exposed to water, there is no teaching or suggestion of incorporating efficient sodium bicarbonate into a paper substrate that would allow the paper substrate to remove odors from adjacent articles in surrounding environment.

There remains room for variation and improvement in the art of deodorizing paper substrates that facilitate the deodorization of other articles.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended Figures in which:

FIG. 1 is a first embodiment of a deodorizing substrate in the form of a creped paper positioned within an optional holder.

FIG. 2 is a partially exploded view of the substrate as seen in FIG. 1 setting forth multiple paper plies of the deodorizing substrate.

FIG. 3 is a view in partial section of a porous pouch which contains therein a confetti type arrangement of a deodorizing paper substrate.

FIG. 4 is a close up view of a deodorizing paper substrate showing a distribution of deodorizing particles which are bound to the paper substrate.

FIG. 5 is a deodorizing article in the form of a container containing therein a deodorizing paper substrate.

FIG. 6 is a deodorizing paper substrate as shown in the form of a shoe insert.

FIG. 7 is a deodorizing substrate seen in the form of a multi-purpose card.

FIG. 8 is a paper deodorizing substrate shown in the form of a hanger card.

FIG. 9 is a deodorizing substrate seen in the form of a clothes hanger sleeve.

FIG. 10 illustrates a vacuum bag storage system containing therein a paper deodorizing substrate.

FIG. 11 is a portable housing containing therein a replaceable paper deodorizing substrate that is suitable for use for damp clothing articles.

FIG. 12 is a graph setting forth titration data to measure baking soda amounts present on the various paper substrates.

FIGS. 13 and 14 are graphs setting forth the performance of various types of odor reducing substrates with respect to shoe odor.

FIG. 15 illustrates a paper substrate deodorizing article in the form of a deodorizing towel.

FIGS. 16A and 16B are optical micrographs of a surface of a paper with sodium bicarbonate which forms a sheet like coating around the paper fibers (16A) and a control sheet (16B).

Repeat use of reference characters in the present specification and drawings is intended to represent the same or analogous features or elements of the invention.

### DETAILED DESCRIPTION OF REPRESENTATIVE EMBODIMENTS

Reference will now be made in detail to embodiments of the invention, one or more examples of which are illustrated in the drawings. Each example is provided by way of explanation of the invention, and not meant as a limitation of the invention. For example, features illustrated or described as part of one embodiment can be used with another embodiment to yield still a third embodiment. It is intended that the present invention include these and other modifications and variations.

It is to be understood that the ranges mentioned herein include all ranges located within the prescribed range. As such, all ranges mentioned herein include all sub-ranges included in the mentioned ranges. For instance, a range from 100-200 also includes ranges from 110-150, 170-190, and 153-162. Further, all limits mentioned herein include all other limits included in the mentioned limits. For instance, a limit of up to 7 also includes a limit of up to 5, up to 3, and up to 4.5.

As used herein, terms “deodorizing substrate” and “deodorizing paper” refer to materials having a cellulosic content in which an effective deodorizing amount of sodium bicarbonate is present within the cellulosic fibers. The various embodiments described herein have been found useful for removing odors from articles and from the ambient environment. The substrates will work within a sealed environment for deodorizing an article. The substrates are also useful for removing odors from an article such as a shoe even when the shoe and deodorizing paper substrate are exposed to ambient conditions.

In accordance with one aspect of this invention, it has been found that applying a 10%-15% by volume of baking soda to a paper web, in an aqueous solution heated to about 110°-150° F., using a conventional paper size press can be used to form a paper substrate having between 3%-6% by weight of sodium bicarbonate present in the end paper substrate product. The 3%-6% loading levels are achieved using a single pass through a paper size press. Greater quantities of sodium bicarbonate up to about 12%-15% by weight can be achieved by using multiple passes through a size press, subsequent spraying of aqueous solutions and baking soda, or in combination with other application or production techniques as described herein.

The fiber content for the paper include a 83% softwood mix of Western North America species and 17% Maplewood hardwood fibers. The fibers were bleached to a brightness of 90. While the fiber content was useful for the development of a paper substrate having the indicated levels of baking soda incorporated therein, is believed that a wide variety of paper making fiber blends and content can be used including fiber content that may increase the ability of baking soda to be incorporated into the paper substrate.

Rolls of paper were manufactured having widths between 22 inches to 48 inches and basis weights ranging from 108# to 130# per 3000 square feet. Sodium bicarbonate was applied as an aqueous solution of 10% and 15% by volume at temperatures between 110° to 150° F. at the size press. For samples in which the cyclodextrin was added, 4% by volume loadings were used and also applied at the size press. Conventional sizing agents were also applied in the paper manufacturing process at loading levels of 2 pounds per ton. For samples where a 15% solution of baking soda was applied at the size press, the sizing agents were reduced by half.

As seen in reference to FIGS. 12-14, comparative data of several different types of a deodorizing paper substrate were made. Basis weight papers of 108#, 120# and 130# papers were evaluated using a size press application of 10% to 15% sodium bicarbonate by volume. As seen in reference to FIG. 12, salt extraction and vinegar extraction analysis of the formed paper substrates indicate that between 3%-6% of sodium bicarbonate level can be achieved using a single size press path in the paper making process. As indicated in the figures, where a 15% solution of sodium bicarbonate in the size press, the amount of conventional sizing agents used with the paper were reduced by half.

It is noted that 3%-6% levels of incorporation of sodium bicarbonate into the paper substrate may create a yellow discoloration and streaking to a bleached paper. While discoloration may be unsuitable for certain paper products, it has been found that the resulting paper substrate offers unexpected odor absorbing qualities. Further, the paper substrate lends itself to subsequent processing into a variety of useful particles adapted for use as a deodorizing article within a number of specialized environments.

The use of a size press to apply solutions or mixtures to a paper web are well known as set forth in U.S. Pat. Nos. 5,401,315, and 7,381,300 the disclosures of which are incorporated herein by reference for all purposes. As conventional within the art, a size press may be used to apply a coating to either one or both surfaces of a paper web. To the extent it is desirable to maximize the amount of sodium bicarbonate present in a resulting paper substrate, it is advantageous to coat both sides of a paper with the aqueous solution of sodium bicarbonate. As seen in reference to FIG. 12, a variety of different basis weight papers may be used to provide a substrate having significant loading levels of sodium bicarbonate incorporated into the paper. So as long as the paper is cable of subsequent handling and forming into desired particles, it is believed that any commercial grade basis weight paper or paper board may be used to form a deodorizing substrate. Sample papers may include conventional office paper grades, coated and uncoated papers, single or multi-ply tissues, various creped papers, paper towels, all in various basis weights and thicknesses.

Various basis weight papers may also be combined to form multi-ply sheets with one or more plies having been treated with sodium bicarbonate. The formation of multi-ply paper can be done using conventional ply forming techniques. Aqueous solutions of a poly-vinyl acetate adhesive have been found useful to laminate sheets of deodorizing paper. The use of an adhesive does not seem to impair the deodorizing capabilities of the paper. Multiple passes through a size press may also be used to increase the amount of sodium bicarbonate present within the paper substrate and additional amounts of sodium bicarbonate can be applied by spray application either during an on-line paper forming step or applied as a separate step during a converting process.

It is believed that when using a size press to apply the sodium bicarbonate, varying degrees of penetration of sodium bicarbonate into the thickness of the paper can be achieved depending upon the basis weight of the paper, paper thickness, moisture content of the paper fiber characteristics and the texture of the paper. It has been found that any conventional grade of baking soda may be used by the size press to apply the sodium bicarbonate. To the extent the sodium bicarbonate is dissolved, the initial particle size of the baking soda is not a critical factor other than the ease of dissolving the sodium bicarbonate within the aqueous solution. While the aqueous solution was frequently maintained at a temperature of 110° F. for safety reasons, increasing the temperature of an aqueous solution does increase the volume percent of baking soda that can be brought into the size press solution. Accordingly, the increased volume can be expected to increase both the amount of sodium bicarbonate applied in the sizing press step as well as the amount retained within the paper substrate.

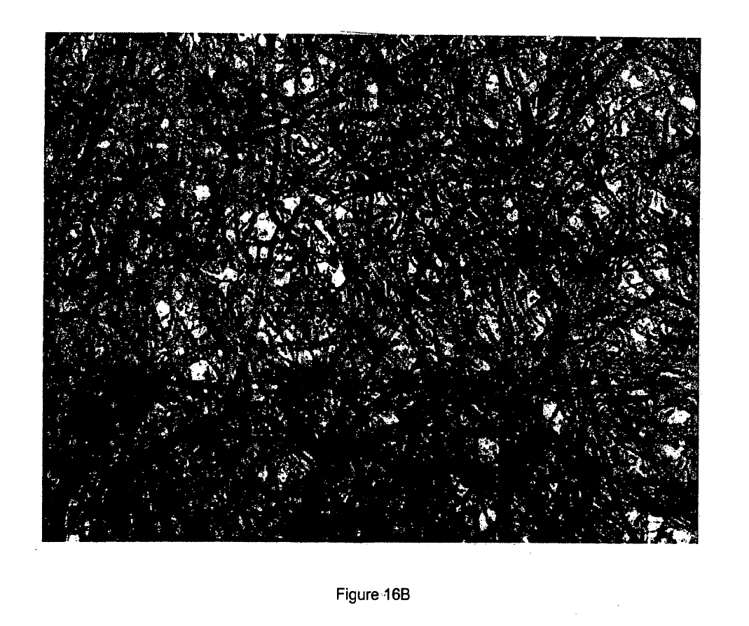

Optical micrographs as seen in FIG. 16A and 16B (control) of the sodium bicarbonate containing paper substrates revealed, in comparison with controls, a reflective, blanket-like coating present within the fibers. While not wishing to be limited by theory, it is believed that the blanket like coating comprises a thin layer of the sodium bicarbonate and which is held within the paper fibers through a combination of electrostatic bonds and hydrogen bonding with the paper fibers.

At the loading levels using a single pass of a size press which achieved a 6.12% by weight of sodium bicarbonate, the sodium bicarbonate substrates have been found highly resistant to the release of the sodium bicarbonate. Simple tests using paper substrates which are agitated by shaking or tapping the substrates against a hard surface released no discernable powder residue. Similarly, rubbing the sodium bicarbonate containing substrate against a dark fabric did not result in any visible transfer of residue from the substrate to the fabric. A similar analysis using hand sheets of paper having a 15% by weight of baking soda applied to the sheet exhibited similar properties of resistance to release of the sodium bicarbonate.

The sodium bicarbonate containing substrate has been found to have a synergistic effect on odor removal. Subjective smell tests have demonstrated that a given quantity of a sodium bicarbonate containing paper substrate is more effective at odor removal during a 65 hour interval than a similar amount of loose sodium bicarbonate.

It is further believed that the quantities of sodium bicarbonate which are incorporated within the various paper substrates described herein have additional functionality as an effective desiccant. The desiccant properties are believed to enhance the odor removal capabilities. Test results demonstrate that the sodium bicarbonate substrate will absorb significantly greater quantities of moisture than untreated control papers.

By way of an example, a hand sheet of a 175 gram per square meter of a creped paper having an addition of 15% sodium bicarbonate by weight, when exposed in a moist, odoriferous environment, had a weight change increase of 235% in comparison to a similar control paper exhibiting less than a 15% weight increase.

The desiccation properties are believed to further enhance the odor absorbing capabilities for many applications. Ambient moisture within an environment may serve as a vector for objectable odors. As such, as the sodium bicarbonate containing substrate attracts moistures, any odors contained within moisture are also drawn into the substrate, allowing the sodium bicarbonate to bind and neutralize such malodors. Further, by removing excess moisture, the low moisture conditions helps prevent bacteria from growing and producing odorous by-products.

Accordingly, the present invention envisions the inclusion of other desiccant materials besides sodium bicarbonate. The inclusion of other desiccants such as silica gel, dehydrated gels, clay, synthetic zeolite, calcium oxide and calcium sulfate can be utilized in the substrate to increase the odor absorbing qualities for the substrate by increasing the moisture absorbing qualities.

An additional advantage of the paper substrate containing sodium bicarbonate is the ability to also function as an anti-microbial product that also has a deodorizing function. Such a substrate is believed useful on low basis weight materials such as paper hand towels having a basis weight of between about 14 to 25 gsm. Incorporation of baking soda levels of between 3%-15% in a paper hand towel product provides a paper substrate that, following use, is resistant to the growth of bacteria. The pH buffering capabilities of baking soda within the paper are believed to alter the pH levels within a used paper towel product such that bacteria cannot grow on the substrate. As a result, potential contamination via soiled towels is greatly reduced. Further, to an extent the towels are placed into a waste receptacle such as a kitchen garbage can, the used towel will continue to have an odor absorbing ability to maintain a clean scent within the waste receptacle.

For many of the embodiments set forth herein, the sodium bicarbonate coated paper substrate can be formed of multiple paper plies. In some embodiments, all plies may contain sodium carbonate in comparable loading levels. It is also envisioned that the amount of sodium bicarbonate may vary with respect to the various plies depending upon the intended end use of the deodorizing substrate. For instance, for some applications it may be beneficial to have one or more plies which comprise a desiccant paper having silica gel or a similar material. Such papers, if included within a multiple-ply structure, would increase the desiccation properties of the overall structure and thereby increase the odor absorption ability of the deodorizing paper.

It is believed that the loading levels set forth herein of sodium bicarbonate within a paper substrate are novel and offer enhanced attributes not found or suggested within any prior combination of sodium bicarbonate and paper. As noted, the preferred levels of sodium bicarbonate present within the substrate may create a discoloration to a bleached paper. For many paper uses, such discoloration would be unacceptable for certain consumer end products. However, as set forth herein, the ability of paper to serve as a carrier for an odor reducing amount of sodium bicarbonate has great utility as a deodorizer.

While the application of sodium bicarbonate to the size press is one useful technique for forming the deodorizing substrate, is believed that other processes and methods can be used to provide an equivalent structure. For instance, incorporation of baking soda into a paper making slurry is possible as set forth in U.S. Pat. No. 7,462,260, assigned to Kimberly Clark Worldwide and which is incorporated herein by reference. The loading levels disclosed in the reference are believed to be significantly less than the amounts utilized in the present invention.

It is also possible to dispense sodium bicarbonate as a solution slurry to a paper web followed by subsequent drying and or optional crepeing steps. One such system is disclosed in U.S. Pat. No. 6,701,637 to Lindsay et al. and which is incorporated herein by reference.

Particles of baking soda can be applied as a powder coating unto a paper web having a coated substrate. As set forth in U.S. Pat. No. 6,355,330 to Koslow et al. and which is incorporated herein by reference, a pressure roller can be used to embed particulates into a softened coating surface present on the paper. While not a preferred method, it is believed that an equivalent substrate can be provided by such a method. Applicant's process of applying baking soda at the size press does not require any type of binder material to be utilized. Accordingly, Applicant's binder free application of sodium bicarbonate within deodorizing substrates and which avoids the undesired shedding or release of baking soda from the finished product is an improvement within the art.

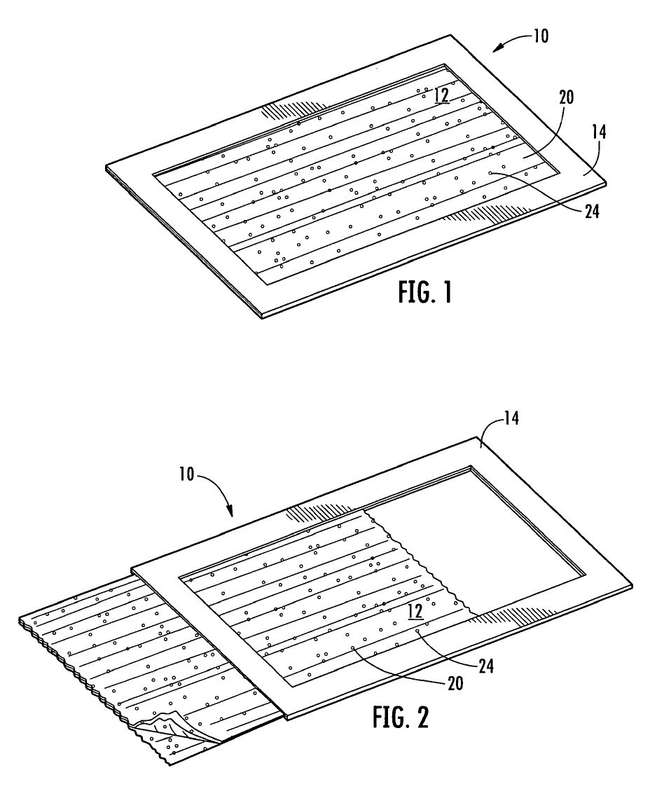

The deodorizing substrates 10 or 12 seen in FIGS. 1 and 2 may also include a cyclodextrin 24 incorporated therewith. In other exemplary embodiments, the cyclodextrin 24 is not present at all in the deodorizing substrate 10. The cyclodextrin 24 used may be α, β, or γ cyclodextrin 24 in certain exemplary embodiments. The cyclodextrin 24 that is selected may be complexed, uncomplexed, or a mixture of both. The cyclodextrin 24 may be provided in a percentage of from 1%-10% of the weight of the total of the cyclodextrin 24 and deodorizing particles 20. In yet other exemplary embodiments, the cyclodextrin 24 is provided from 1%-10% by weight percentage of the total of the cyclodextrin 24 and the total of the cyclodextrin 24, base sheet 12, and deodorizing particles 20. The cyclodextrin 24 may be of any size. For example, the cyclodextrin 24 is 100 microns in accordance with one exemplary embodiment. The cyclodextrin 24 may function to add odors to an article to be deodorized. Further, the desiccant properties of baking soda in a low basis weight paper tissue or paper towel product will also enhance the ability of product to absorb moisture from an individuals' hands or body. Once disposed, the pH of the deodorizing substrate helps prevent the growth of any bacteria that may have been transferred to paper substrate. The cyclodextrin 24 is used to trap, capture and/or remove odors of the item 16 such as those caused by cigarette smoke or perfume.

The present invention provides for a deodorizing substrate. The deodorizing substrate 10 includes a plurality of deodorizing particles 20 that function to absorb, neutralize, or otherwise remove odiferous molecules associated with an item to improve the smell of the item. The deodorizing substrate 10 can be incorporated into a variety of deodorizing articles for use in specific environments.

Evaluation of the commercial grade papers having baking soda incorporated therein has indicated that the papers remain printable and can be scored and folded to various configurations without any perceived degradation of the standard paper qualities.

FIG. 1 illustrates a deodorizing substrate 10 in accordance with one exemplary embodiment. The deodorizing substrate 10 has a base sheet 12 that is used to carry other portions of the deodorizing substrate 10. The base sheet 12 may be made of a variety of materials. For example, the base sheet 12 may be made of paper. Optionally shown in FIG. 1 is a holder 14 in the form of open mat adapted for receiving base sheet 12 within an interior of mat 14. The use of the supportive mat 14 facilitates the use of a thinner paper substrate for the base sheet 12. As seen in reference 2, FIG. 2, base sheet 12 can be a multi-ply sheet in which one or more plies carry deodorizing particles 20.

The deodorizing substrate 10 includes a plurality of deodorizing particles 20. The deodorizing particles 20 may be made of a variety of different materials. For example, in one exemplary embodiment the deodorizing particles 20 are made of sodium bicarbonate. Sodium bicarbonate is sometimes known as sodium hydrogen carbonate, sodium acid carbonate, baking soda, and bicarbonate of soda. In other exemplary embodiments, the deodorizing particles 20 can be potassium bicarbonate. In yet other arrangements, the deodorizing particles 20 may be cyclodextrin and/or zeolite. In still further embodiments, the deodorizing particles 20 may be a combination of sodium bicarbonate and potassium bicarbonate. The deodorizing particles 20 may be from 0.5-600 microns in size, from 6-150 microns, or from 25-50 microns with the majority being 40 microns in accordance with different exemplary embodiments. As noted earlier in the application, where the particulates are selected from sodium bicarbonate and applied to paper from a heated aqueous solution, the resulting coating layer does not form discrete particles.

With reference to FIG. 2, the paper substrate 12 can be positioned within the template 14. However, it is noted that the paper substrate 12 may be selected from a sufficiently thick paper stock such that the substrate 12 having baking soda 20 incorporated therein will function as a stand alone deodorizing member without the need of a supporting template 14. One aspect of some of the embodiments described herein make use of a paper as a carrier for sodium bicarbonate. It has been found that the delivery of sodium bicarbonate on a paper substrate enhances the ability of sodium bicarbonate to deodorize an article.

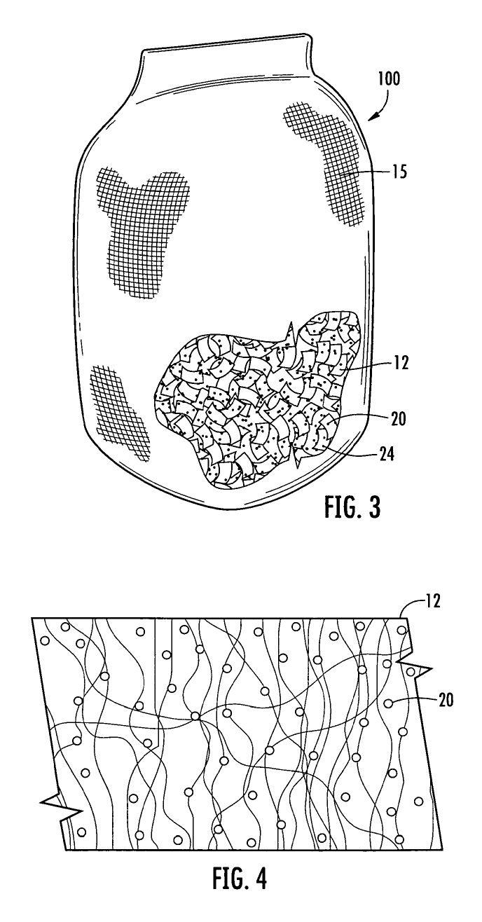

As seen in reference to FIG. 3, an alternative embodiment of the present invention is provided by a pouch 100 having sealed within an interior of the pouch a plurality of confetti like paper substrate 12. The pouch may be formed from a variety of open mesh materials 15 including various types of netting, nonwoven mesh or porous paper similar to that used to form tea bags. The porous material 15 provides the pouch 100 with air flow in communication with the deodorizing paper substrate 12 housed within the pouch. The deodorizing pouch can be used in a wide variety of environments and is compressible when inserted into a desired space. The deodorizing substrate as seen in pouch 100 can be used in a refrigerator, compartments of the refrigerator, inserted into shoes, dresser drawers, interior of automobiles, pet beds, and other locations where a deodorizing substrate is desired.

As seen in reference to FIG. 4, an enlargement of a surface of a paper substrate 12 is set forth showing the sodium bicarbonate particles 20. As previously noted, when the sodium bicarbonate solution is applied by a size press, the sodium bicarbonate appears as a coating across the fibers. The resulting film of particles 20 are firmly secured within the fiber matrix through what is believed to be a combination of electrostatic charges and hydrogen bonding. It is believed that when the dissolved sodium bicarbonate dries within the paper, a film of dried sodium bicarbonate is formed as best seen in reference to FIG. 16.

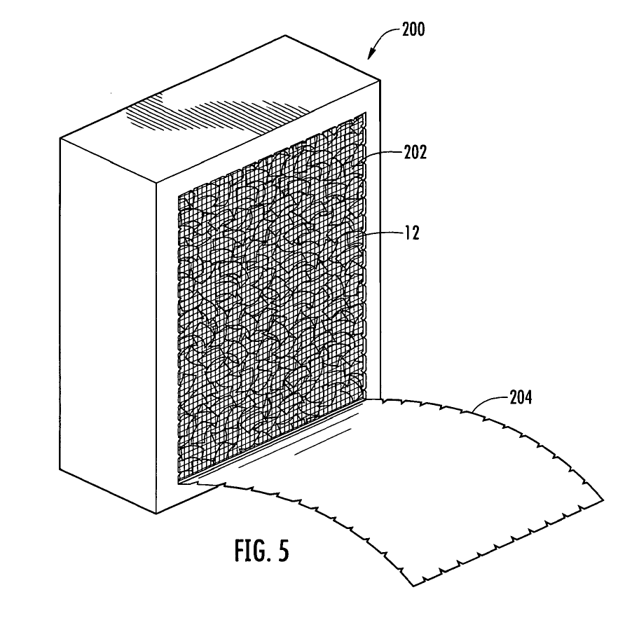

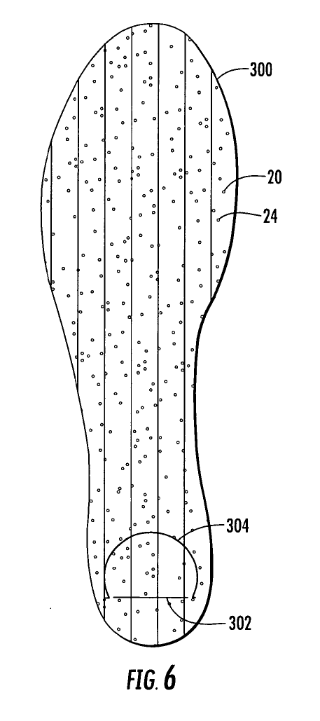

FIG. 5 sets forth a container 200 having at least a portion of one wall defining a porous opening 202 that permits air flow into an interior of the container 200. A plurality of deodorizing paper sheets 12 having sodium bicarbonate therein in the form of a paper confetti, are contained therein. As seen, a flap 204 can be removed to provide communication between the deodorizing paper 12 within the interior of carton 200 and the exterior of the carton 200. This embodiment can be used in a number of environments including refrigerators, freezers and other locations to facilitate the removal of odors.

An additional embodiment of a paper deodorizing substrate seen in reference to FIG. 6, where the deodorizing member is in the form of a shoe insert 300 and which generally conforms to the shape and dimensions of a shoe insole. The shoe insert 300 can be provided from a number of paper grades in either a creped or uncreped paper and provided as a single-ply or a multi-ply configuration. A scored hinge line 302 can be provided within a hill portion of the insert 300. Score line 302 is designed to function as a hinge which allows attachment of a flap portion defined along a perforation line 304. A combination of the hinge and flap permits the user to grasp the insert for easier insertion and removal from a shoe.

It has been found that when a two ply laminated shoe insert 300 incorporated therein a deodorizing amount of sodium bicarbonate 20 between the ranges of about 3% to about 6%, the insert is effective at deodorizing a shoe within 24 hours. The shoe insert 300 may optionally include a cyclodextrin 24 to further assist with deodorizing capabilities.

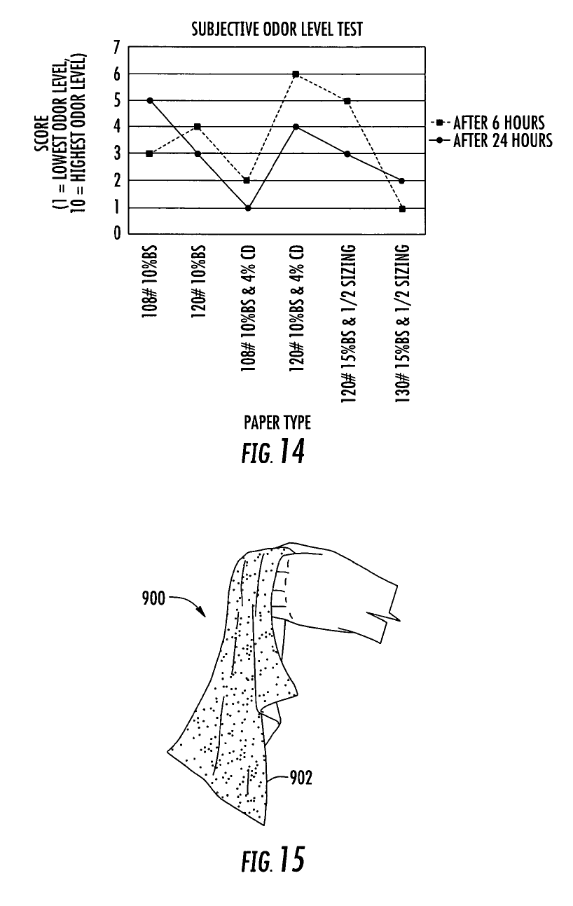

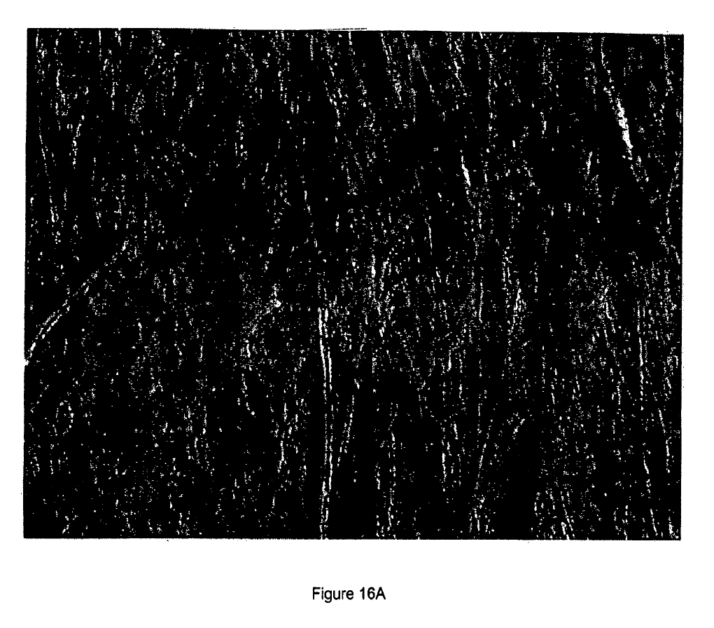

As seen in FIGS. 13 and 14, odor evaluation and odor reduction tests were conducted using the odor analog of 1% isovaleric acid in a shoe. Following the application of the isovaloric acid, a two-ply shoe shaped deodorizing paper substrate was inserted into each shoe. Participants ranked the level of odor on a scale of 1 to 10, with 10 being the strongest/worst odor and 1 being the least amount of odor. The data set forth in FIGS. 12 and 13 sets forth evaluations of the participants on a scale of least odoriferous (1) to the most odoriferous (10). As noted in the Figures various grades of paper were evaluated based upon coating levels of sodium bicarbonate with some samples including a 4% by volume application of cyclodextrin. As seen in the date from FIG. 13, using baking soda alone or in combination with a cyclodextrin, the substrates are capable of achieving significant odor reduction.

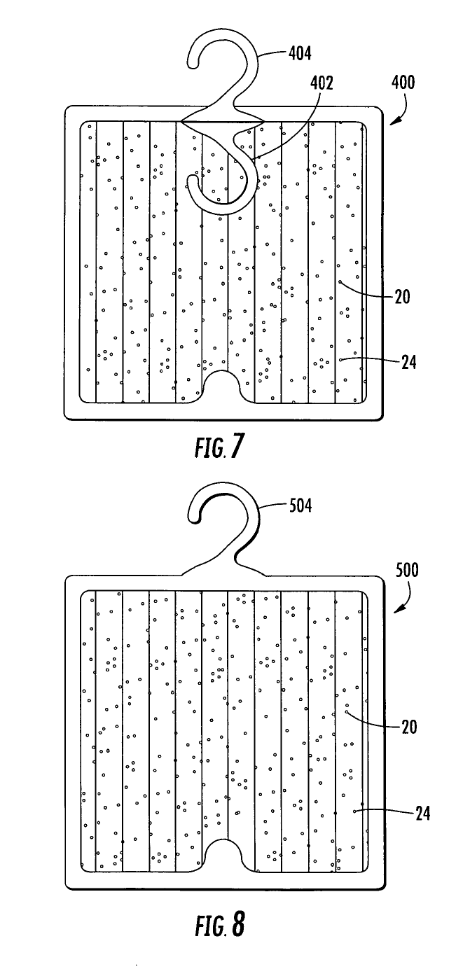

Set forth in FIG. 7 is an additional embodiment of a paper deodorizing member seen in the form of a multipurpose card 400. As illustrated, the card may initially define, within an interior space, a perforated section 402 corresponding to a shaped hook 404. Upon breaking the perforations, the hook portion 404 can be folded such that the card can be hung on an object such as a closet rail, a coat hanger, within the interior of a garment bag or any other suitable environment where a deodorizing hanging substrate is desired. It is noted that the hanger card of FIG. 7 can also be used as a flat card for multiple applications as a general deodorizer.

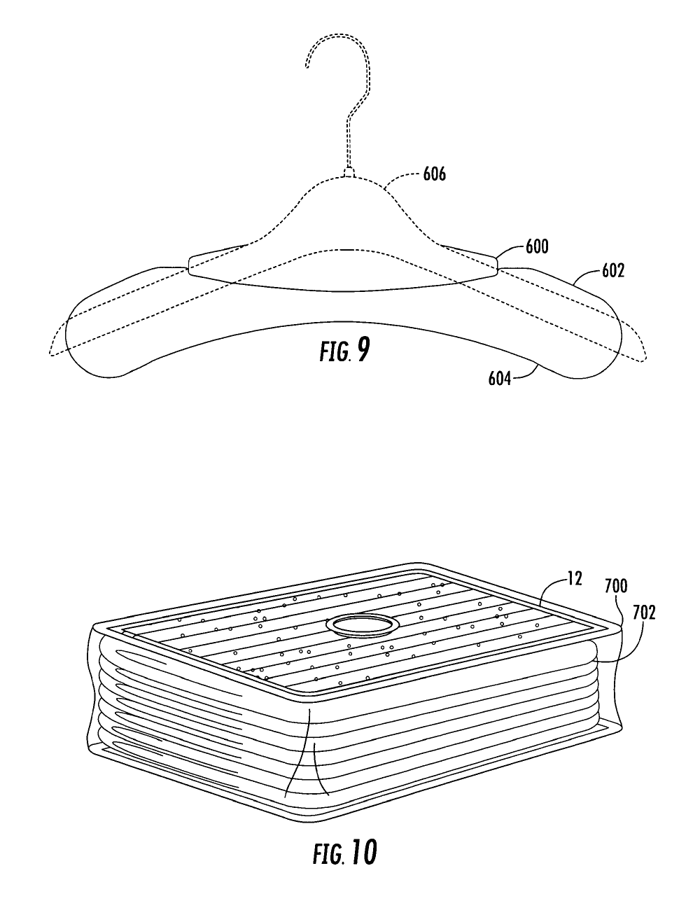

In reference to FIG. 8, a hanger card 500 is provided of a deodorizing paper substrate in which a deodorizing paper has an integral hook 504. While the dimensions and shape may vary, the hanger card 500 is useful for placement on closet rods, within garment bags, attached to traditional hangers and used in other environments where it is useful to hang a deodorizing member.

In reference to FIG. 9, a coat hanger skirt 600 is provided of a deodorizing paper substrate. As illustrated, the hanger skirt defines overlapping edge portions 602 to 604 which allow for a hook of a conventional clothes hanger 606 to be inserted there through. The resulting deodorizing substrate of hanger skirt 600 is placed on opposite sides of the clothes hanger 606 and will be in intimate contact with any garment placed upon the hanger. While the illustrated embodiment shows a hanger skirt 600 which is sized to conform to the general dimensions to a hanger, the bottom edges and sides 602 and 606 of the hanger skirt could be of greater length so as to provide a larger deodorizing surface to a garment such as a jacket or coat.

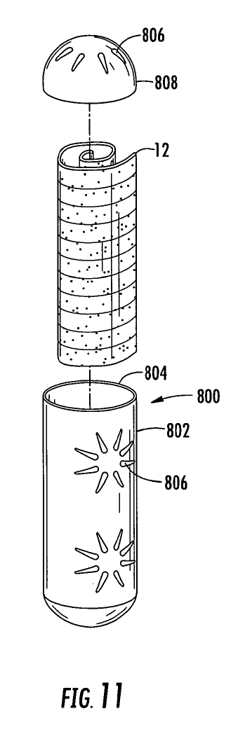

As seen in reference to FIG. 10, the various deodorizing members described herein may be used within the interior of a vacuum storage bag 700 to provide deodorizing capabilities to stored materials. Typically, such storage bags are used to store clothing items 702. The inclusion of a deodorizing paper substrate member 12 helps maintain a fresh scent to the stored articles. Further, the deodorizing member helps prevent the transfer of a plastic odor from the storage bag material to the articles contained therein. To the extent the storage bag is sealed and maintained under a vacuum, the deodorizing properties of the deodorizing member can continue for the extended storage period since the total volume of odors is small. An additional advantage of the deodorizing member of the preferred embodiments is the desiccation properties of deodorizing member. The ability of the deodorizing member to further absorb excess moisture helps to preserve and safe guard the stored articles and thereby removes any additional source of odors and odor migration.

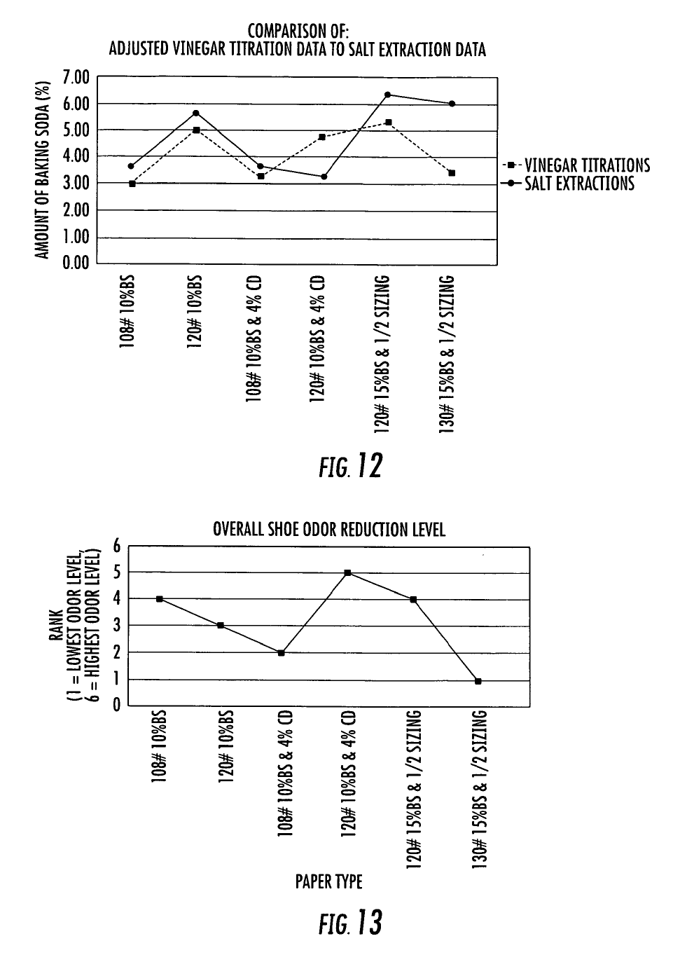

As set forth in FIG. 11, an additional embodiment of an odor control device 800 is seen in the form of a porous ridged tube 802 that can be made of plastic, metal, or a similar rigid material. The tube has at least one accessible opening 804 that allows the insertion of a paper deodorizing substrate 12 to be placed within the interior of the tube. As noted, the walls of the tube and/or the end caps 808 of the tube define a plurality of the vent like openings 806 to allow for the passage of air. This particular embodiment is useful for providing deodorizing to a gym bag or laundry bag where damp articles may be present. The tube housing protects the enclosed paper deodorizing substrate 12 from direct contract with moisture but provides sufficient air flow that deodorization can occur.

As seen in reference to FIG. 15 an additional embodiment of a paper based deodorizing substrate 900 is provided as seen in the form of a disposal paper sheet 902. Preferably, the paper sheet has absorbent qualities that can be used as a towel for deodorizing a damp pet. Use of a sodium bicarbonate deodorizing substrate to further “towel dry” a damp pet can be effective in removing additional odors. In addition, deodorizing member seen in FIG. 15 can easily be cut to size for various applications and needs. The sheet can also be used to deodorize areas such as damp upholstery or carpets. The paper sheet 902 can be provided from a low basis weight material such as a traditional paper towel that might be provided on a roll for consumer use. In addition, heavier basis weight paper can be used to form the paper sheet 902 which can be more substantial and used more like a fabric and/or laundry towel.

A deodorizing substrate 10 or a deodorizing sheet of paper 12 as seen in reference to FIGS. 1 and 2 may also be used as an insert with food packaging. Particular, it is believed that the substrate is useful for inclusion of packaging of fresh meat and seafood to reduce odors associated with the food packaging. For instance, a deodorizing paper substrate 12 may be placed in a non-food contact portion of fresh packaged meat or seafood packaging to absorb odors.

It is readily understood and appreciated by those having ordinary skill in the art that to maintain the deodorizing properties of the paper base deodorizing substrates and products described herein are best packaged and stored in some type of air tight packaging until used. Air tight packaging prevent the loss of deodorizing capabilities brought about by exposure to the ambient environment.

While the present invention has been described in connection with certain preferred embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

What is claimed:

1. A deodorizing substrate, comprising:
a base sheet of a cellulosic substrate; and
a plurality of deodorizing particles carried by the cellulosic substrate, said particles comprising at least about 3%-6% of sodium bicarbonate.

2. The deodorizing substrate as set forth in claim 1, wherein the deodorizing particles further include an additional additive selected from a group consisting of potassium bicarbonate, cyclodextrin, zeolite, and combinations thereof.

3. The deodorizing substrate as set forth in claim 1, wherein some of the deodorizing particles are sodium bicarbonate and wherein some of the deodorizing particles are cyclodextrin.

4. A deodorizing substrate comprising;
a base sheet of a cellulosic substrate; and
deodorizing particles carried by cellulosic substrate, the particles comprising between about 3% to about 15% of sodium bicarbonate.

5. The deodorizing substrate according to claim 1, wherein said deodorizing substrate is in the form of a shoe insert.

6. A deodorizing substrate according to claim 1, wherein said substrate is in the form of a sleeve adapted for insertion along opposite sides of a clothes hanger.

7. A deodorizing substrate according to claim 1, wherein said cellulosic substrate additionally contains a desiccant.

8. A process of manufacturing a deodorizing substrate comprising;
providing a cellulosic web of material;
applying to said web a solution of between about 10% to about 15% by volume of sodium bicarbonate;
drying said cellulosic web and said solution, thereby providing a cellulosic web containing an effective amount of odor reducing sodium bicarbonate ranging from about 3% to about 15%, the sodium bicarbonate being bound to the cellulosic fibers.

9. A deodorizing substrate consisting essentially of:
a base sheet of a cellulosic substrate; and
a plurality of deodorizing particles carried by cellulosic substrate, with particles comprising at least about 3%-6% of sodium carbonate, more preferably at least about 4%-8% of sodium bicarbonate, and more preferably about 6%-12% of sodium bicarbonate and more preferably about 12%-15% of sodium bicarbonate.

10. The deodorizing substrate according to claim 1, wherein said particles in the cellulosic substrate are in the form of a film coating.

11. The process according to claim 8, wherein said step of drying said solution forms a film of sodium bicarbonate within the cellulosic web.

12. The deodorizing substrate according to claim 1, wherein said deodorizing substrate is contained within a rigid air permeable housing.

13. A deodorizing substrate according to claim 1, wherein said deodorizing substrate further comprises of multiple plies of a cellulosic substrate.

14. The process of according to claim 8, wherein said step of applying a solution of sodium bicarbonate further comprises heating the solution of sodium bicarbonate to a temperature of between about 110° F. to about 150° F.

15. A deodorizing substrate according to claim 1, wherein said deodorizing substrate is in the form of a paper towel having a basis weight of between 14-25 gsm.

16. A deodorizing substrate according to claim 13, wherein at least one of said plies further comprises a desiccant paper.

17. The process according to claim 8, wherein said step of applying to said web a solution of sodium bicarbonate further comprises using a size press to apply the solution.

18. The process according to claim 8, wherein said step of applying a solution to said web further comprises spraying said solution onto said web.
